(19) United States
(12) Patent Application Publication
(10) Pub. No.: US 2025/0127693 A1
(43) Pub. Date: Apr. 24, 2025

(54) Materials for Desensitization and Targeted Drug Delivery to Teeth and Gums

(71) Applicant: South Dakota Board of Regents, Pierre, SD (US)

(72) Inventor: Grigoriy Sereda, Vermillion, SD (US)

(21) Appl. No.: 18/919,734

(22) Filed: Oct. 18, 2024

Related U.S. Application Data
(60) Provisional application No. 63/591,192, filed on Oct. 18, 2023.

(51) Int. Cl. A61K 8/19; A61K 8/02; A61K 8/21; A61K 8/34; A61K 8/42; A61K 8/43; A61K 8/63; A61Q 11/00

(52) U.S. Cl. CPC A61K 8/19; A61K 8/025; A61K 8/0254; A61K 8/21; A61K 8/34; A61K 8/347; A61K 8/42; A61K 8/43; A61K 8/63; A61Q 11/00

(57) ABSTRACT

Disclosed herein are dentifrices with low pH-triggered release of pharmacons and other materials to more effectively adhere to the tooth and associated surfaces. The various compositions and methods include novel dental compositions that release materials such as fluoride ions, eugenol, pharmacons or other medicinal payloads under physiologically low pH to extend the material lifespan on the teeth and gums of the user.

### CROSS-REFERENCE TO RELATED APPLICATION(S)

This application claims the benefit under 35 U.S.C. § 119 (e) to U.S. Provisional Application 63/591,192, filed Oct. 18, 2023 and entitled “Materials for Desensitization and Targeted Drug Delivery to Teeth and Gums,” which are hereby incorporated herein by reference in their entireties.

### GOVERNMENT SUPPORT

This invention was made with government support under Grant No. 2342430, awarded by the National Science Foundation. The government has certain rights in the invention.

### FIELD

The various embodiments herein relate to the field of dental management, including teeth and gums. More specifically, the various embodiments relate to dentifrices for application to teeth and gums, such as toothpastes, flosses, and medical ointments for treating dental diseases, or providing oral medication through applications to the teeth and gums.

### BACKGROUND

Dental diseases are one of the most common public health problems among all communities affecting people throughout their lifetime, causing pain, discomfort, and disfigurement. According to the estimations from the Global Burden of Disease Study 2016, oral diseases affected half of the world's population (3.58 billion people) with dental caries (tooth decay) in permanent teeth (Vos, T. et al., Lancet 2017, 390, 10100, 1211-59). More than half of children in the United States have at least one cavity or filling, which increases to 78% among teenagers (Sachdev, J. et al., Int. J. Clin. Pediatr. Dent 2016, 9, 1, 15-20). The ultimate presence of these dental diseases is the damage of the tooth enamel (dental caries), inflammation of the periodontal gums (gingivitis), and the supporting periodontal tissues (periodontal diseases). Prevention, control, and responsive treatment are the most important actions for maintaining healthy teeth. Fluoride is a well-known and daily used anti-demineralization agent delivered externally by toothpastes to prevent dental diseases such as dental caries. However, the major issue with fluoride-containing dentifrices is their short action time due to their dilution and clearance by saliva. Further, selective delivery of fluoride to the areas where the teeth need them the most would allow for the substantial decrease of the total amount of fluoride and alleviate potential issues with its toxicity. A commonly used drug chlorhexidine causes undesired side effects such as tooth staining, taste alteration in mucosal irritation (Periodontology 2000 2008, 48 (1), 54-65). Bio-adhesive selective drug delivery systems will allow scientists to overcome these shortcomings. The toothpaste market is the largest section of the oral care market. In 2018, the toothpaste market was worth USD 26.1 billion and poised to reach USD 37.0 billion by 2024 (Global $36+ Billion Toothpaste Market, 2024: Growth, Trends and Forecast Analysis from 2019—ResearchAndMarkets.com. https://www.businesswire.com/news/home/20190509005698/en/Global-36-Billion-Toothpaste-Market-2024-Growth).

Hydroxyapatite (HA) [Ca₁₀(PO₄)₆(OH)₂] is the most common calcium phosphates phase, which has been substantially investigated in several biomedical applications due to its excellent biocompatibility, bioactivity, and chemical composition similar to the natural bones and teeth (Verma, G. et al., Ceram. Int 2013, 39, 8, 8995-9002; and Neelakandeswari, N. et al., Synth. React. Inorg., Met. Org., Nano-Met. Chem 2011, 41, 5, 513-6). HA particles are widely utilized as bone substitutions (Dorozhkin, S. V., Materials 2013, 6, 9, 3840-2), dental materials (Tschoppe, P. et al., J. Dent 2011, 39, 6, 430-7), gene carriers (Izuegbunam, C. L. et al., Nanoscale Advances 2021, 3, 11, 3240-50), drug delivery carriers (Mondal, S. et al., Wiley Interdiscip. Rev.: Nanomed. Nanobiotechnol 2018, 10, 4, e1504. Epub 2017 Nov. 23), ion exchangers (Feng, Q. L. et al., Thin Solid Films 1998, 335, 1-2, 214-9), antibacterial agents (Ciobanu, C. S. et al., Nanoscale Res. Lett 2012, 7, 324, 1-9), and catalysts (Sadat-Shojai, M. et al., Acta Biomater 2013, 9, 8, 7591-7621). Hydroxyapatite nanoparticles (HA NPs), along with other dentinal desensitizers, have been used in oral care products such as dentifrices and mouthwashes to temporarily alleviate or eliminate tooth discomfort by preventing the opening of dentinal tubules that extend through the dentin to the pulp ((Clark, D. et al., Int Dent J 2016, 66, 249-56; Vano, M. et al., Quintessence Int. Berl. Ger 2014, 45, 703-11). In a 2016 in vitro study, HA NPs have shown higher desensitizing potential than commercially available, well-established desensitizing compositions (Kulal, R. et al., J. Clin. Diagn. Res 2016, 10, ZC51-4). HA NPs with bactericidal effects have been of great interest in minimizing the formation of dental cavities (Aljabo, A. A. et al., J. Mater. Sci. Eng. C 2016, 60, 285-292). Preventing dental caries includes inhibiting or killing pathogenic oral bacteria, including Streptococcus mutans, in the lower layer of the oral biofilm. To date, several studies have determined the ability of various nanoparticles, including HA, to inhibit or kill these bacteria (Liao, J. et al., Trans Tech Publ 2007, 299-302). Adhesion of particles to dentin appears to be the initial step in occlusion, followed by other contributing mechanisms such as particle aggregation, which triggers the deposition of more insoluble materials in the tubules and particle transport to the tooth surface (Rashwan, K. et al., Chapter 5 in Book Nanotechnology: Delivering on the Promise, Vol. 2, ACS Symposium Series, Vol. 1224, ISBN13: 9780841231467, 2016, 95-105). The key advantage of HA NPs is their structural resemblance to the hydroxyapatite component of dentin and enamel, enabling their bioactivity and biocompatibility (Pepla, E. et al., Ann. Stomatol (Roma) 2014, 5, 108-14). Earlier it was shown that hydroxyapatite nanoparticles can be delivered to the tooth surface by functionalized silk dental floss (Sereda, G. et al., Am J Dent 2019, 32, 3, 118-23). However, the potential of HA and other materials adhering to dentin to carry and release pharmaceuticals at the tooth surface remained unexplored. Further, more basic micro- and nanoparticles of vaterite or amorphous calcium carbonate with a better pH-buffering capability than HA have been so far unavailable for dental drug delivery applications due to their low thermodynamic stability (Boyjoo, Y. et al., J Mater Chem A 2014, 2, 14270-8).

There is a need in the art for improved compositions and methods for desensitization and targeted drug delivery to teeth and gums, including, for example, non-toxic materials able to adhere to dentin and gums, occlude dentinal tubules, hold pharmacons at the surface of dentin, and release them on-demand where the tooth or gum tissue need them most. The targeted delivery of pharmacons will significantly decrease overall patient's exposure to a drug and, therefore, reduce adverse side effects.

### BRIEF SUMMARY

Discussed herein are various compositions and methods of use disclosed or contemplated herein, which include novel dental compositions that release materials such as fluoride ions, eugenol, pharmacons or other medicinal payloads under physiologically low pH to extend the material lifespan on the teeth and gums of the user. The compositions efficiently adhere to the surface of human dentin and can occlude dentinal tubules. The compositions may include various HA and calcium carbonate-based particles to carry and release eugenol, chlorhexidine, fluoride anions or other pharmacons at the surface of dentin or enamel in a time-, pH-, and enzyme-dependent manner. The low stability of this type of material in a range of pH 4-6 is essential for the targeted drug delivery to the tooth areas mostly affected by the decay triggered by bacteriogenic acids.

In Example 1, a dentifrice comprising a carrier material, and the dentifrice has a pH between 6 and 8.

Example 2 relates to the dentifrice according to Example 1, wherein the carrier material is comprised of one or more of calcium carbonate microsphere, a calcium carbonate nanosphere, a calcium carbonate/hydroxyapatite microplatelets, a calcium hydroxyapatite, or a mesoporous hydroxyapatite particle, and wherein the carrier material is used to carry fluoride ions, eugenol, menthol or a pharmacon.

Example 3 relates to the dentifrice according to Example 2 wherein the carrier material further comprises a phospholipid or a phosphoprotein.

Example 4 relates to the dentifrice according to Example 2, wherein the carrier material is casein coated.

Example 5 relates to the dentifrice according to Example 2, wherein the pharmacon is doxycycline, chlorhexidine, prednisolone, or veratridine.

Example 6 relates to the dentifrice according to Example 1, wherein the dentifrice is an active ingredient in a toothpaste, a dental floss, a toothpick, or a coating on a dental device.

Example 7 relates to the dentifrice according to Example 1, wherein the dentifrice is an application that is applied to teeth, gums, or an interior surface of an oral cavity.

Example 8 relates to a method of administering a cargo to teeth or gums, the method comprising applying a composition with an active ingredient comprising a carrier material and the cargo, wherein the composition has a pH between 6 and 8.

Example 9 relates to the method according to Example 8, wherein the composition is applied to occlude dental tubules, attach to dentin, or attach to gum tissue.

Example 10 relates to the method according to Example 8, wherein the carrier is one or more of calcium carbonate microsphere, a calcium carbonate nanosphere, a calcium carbonate/hydroxyapatite microplatelets, a calcium hydroxyapatite, or a mesoporous hydroxyapatite particle.

Example 11 relates to the method according to Example 8, wherein the carrier further comprises a phospholipid or a phosphoprotein.

Example 12 relates to the method according to Example 11 wherein the carrier is casein coated.

Example 13 relates to the method according to Example 8, wherein the cargo is fluoride ions, eugenol, menthol or another pharmacon.

Example 13 relates to the method according to Example 8, wherein at least a portion of the cargo remains on the teeth or gums for over 10 minutes.

Example 14 relates to the method according to Example 8, wherein at least a portion of the cargo remains on the teeth or gums for over 30 minutes.

Example 15 relates to the method according to Example 8, wherein at least a portion of the cargo remains on the teeth or gums for at least one hour.

Example 16 relates to the method of claim 11 wherein the phospholipid or a phosphoprotein is digested by a matrix metalloproteinase (MMP) enzyme.

Example 17 relates to the method of claim 16 wherein the MMP enzyme is a matrix metalloproteinase-9 (MMP-9) enzyme.

Example 18 relates to a dentifrice composition comprising a casein-coated calcium carbonate microsphere and a cargo, wherein the dentifrice has a pH of about 6-8 and the cargo is fluoride ions, eugenol, or a pharmacon.

While multiple embodiments are disclosed, still other embodiments will become apparent to those skilled in the art from the following detailed description, which shows and describes various illustrative implementations. As will be realized, the various embodiments herein are capable of modifications in various obvious aspects, all without departing from the spirit and scope thereof. Accordingly, the drawings and detailed description are to be regarded as illustrative in nature and not restrictive.

### BRIEF DESCRIPTION OF THE DRAWINGS

FIG. 1 contains SEM images of the 1-4 μm calcium carbonate microspheres prepared by the procedure and shows their high porosity. The particles are not stable in a 2 mg/ml aqueous suspension for more than 2 days were stabilized by pH 7.4 phosphate buffer saline (PBS) and much more efficiently—by a casein shell (7.2 μg casein/1 mg particles). FIG. 1(a) as prepared calcium carbonate microspheres; (b) calcium carbonate microspheres after 2 days in water; (c) calcium carbonate microspheres after 15 days in pH 7.4 PBS; (d) casein-coated calcium carbonate microspheres after 15 days in water.

FIG. 2 contains SEM images of (a) calcium carbonate nanospheres after 7 days in water; (b) casein-coated calcium carbonate nanospheres after 7 days in water.

FIG. 3 is an SEM image of calcium carbonate/hydroxyapatite core-shell Microplatelets.

FIG. 4 contains SEM images of (a) Hydroxyapatite nanoneedles and (b) Hydroxyapatite mesoporous microparticles.

FIG. 5 contains SEM images of (a) Dentin before application of particles; (b) Dentin occluded by casein-coated calcium carbonate microspheres; and (c) Dentin occluded by mesoporous hydroxyapatite microparticles. (d) Enamel after application of casein-coated calcium carbonate microspheres.

FIG. 6 contains SEM images of (a) Dentin occluded by hydroxyapatite nanoneedles; (b) Dentin occluded by casein-coated hydroxyapatite nanoneedles; (c) Dentin occluded by calcium carbonate/hydroxyapatite microplatelets; and (d) Dentin occluded by casein-coated calcium carbonate/hydroxyapatite microplatelets.

FIG. 7 shows the cumulative eugenol release profiles from casein-coated calcium carbonate microspheres in phosphate buffer saline at pH 7.4 (circle line) and pH 5.5 (square line).

FIG. 8 shows the cumulative eugenol release profiles from calcium carbonate/hydroxyapatite microplatelets in phosphate buffer saline at pH 7.4 (square line); cumulative eugenol release profile from casein-coated calcium carbonate/hydroxyapatite microplatelets in phosphate buffer saline at pH 7.4 (triangle line); and cumulative eugenol release profile from casein-coated calcium carbonate/hydroxyapatite microplatelets in phosphate buffer saline at pH 5.5 (circle line).

FIG. 9 shows the cumulative eugenol release profiles from hydroxyapatite nanoneedles in phosphate buffer saline at pH 7.4 (square line) and the cumulative eugenol release profile from casein-coated hydroxyapatite nanoneedles in phosphate buffer saline at pH 7.4 (circle line) and pH 5.5 (triangle line).

FIG. 10 shows the cumulative eugenol release profiles from mesoporous hydroxyapatite particles in phosphate buffer saline at pH 7.4 (square line) and at pH 5.5 (circle line).

FIG. 11 shows the cumulative Fluoride ion release profiles from casein-coated calcium carbonate microspheres in phosphate buffer saline at pH 7.4 (square line); cumulative eugenol release profile from casein-coated calcium carbonate microspheres in phosphate buffer saline at pH 5.5 (circle line) and artificial saliva at pH 6.9 (triangle line).

FIG. 12 shows the cumulative eugenol release profiles from casein-coated calcium carbonate microspheres in phosphate buffer saline at pH 7.4 on dentin (circle line); cumulative eugenol release profile from casein-coated calcium carbonate microspheres in phosphate buffer saline at pH 5.5 on dentin (triangle line); and cumulative eugenol release profile from eugenol-treated dentin (square line).

FIG. 13 shows the cumulative eugenol release profiles from casein-coated calcium carbonate/hydroxyapatite microplatelets in phosphate buffer saline at pH 7.4 on dentin (circle line); cumulative eugenol release profile from casein-coated calcium carbonate microspheres in phosphate buffer saline at pH 5.5 on dentin (triangle line); and cumulative eugenol release profile from eugenol-treated dentin (square line).

FIG. 14 shows the cumulative eugenol release profiles from casein-coated hydroxyapatite nanoneedles in phosphate buffer saline at pH 7.4 on dentin (circle line); cumulative eugenol release profile from casein-coated calcium carbonate microspheres in phosphate buffer saline at pH 5.5 on dentin (triangle line); and cumulative eugenol release profile from eugenol-treated dentin (square line).

FIG. 15 shows the cumulative eugenol release profiles from mesoporous hydroxyapatite particles in phosphate buffer saline at pH 7.4 on dentin (square line); cumulative eugenol release profile from casein-coated calcium carbonate microspheres in phosphate buffer saline at pH 5.5 on dentin (circle line); and cumulative eugenol release profile from eugenol-treated dentin (triangle line).

FIG. 16 shows the synthesis of modified calcium carbonate microspheres and their application for acid-triggered drug delivery.

FIG. 17 shows the cumulative doxycycline release profiles from casein-coated CaCO₃ microparticles in phosphate buffer saline at pH 7.4 (circle line) and pH 5.5 (square line) with an encapsulated drug.

FIG. 18 shows the cumulative chlorhexidine release profiles from casein-coated CaCO₃ microparticles in phosphate buffer saline at pH 7.4 (circle line) and pH 5.5 (square line) with an encapsulated drug.

FIG. 19 shows that chlorhexidine-loaded and casein-coated calcium carbonate microparticles (CHX@CCMP-CAS) significantly suppress growth of colonies of Streptococcus mutan (caries-causing bacteria) comparing with the control (0.02% chlorhexidine), particularly at pH 5.5 when the drug release is triggered.

FIG. 20 is a cumulative chlorhexidine (CHX) release profile of casein from diluted skin milk coated CaCO₃ microparticles in TBS at pH 7.4 (triangle line) and pH 5.5 (square line).

FIG. 21 is a cumulative VTD release profile of casein coated CaCO₃ microparticles in TBS with MMP present (top line) and without MMP-9 present at the physiological 100 ng/ml (bottom line).

### DETAILED DESCRIPTION

The various compositions and methods of use disclosed or contemplated herein include novel dental compositions that release materials such as fluoride ions, eugenol, pharmacons or other medicinal payloads under physiologically low pH to extend the material lifespan on the teeth and gums of the user. Eugenol is used in the dental practice and its well-documented desensitizing, antibacterial (Markowitz, K et al.), and anticancer (Jaganathan, S. et al.) properties, while fluoride is a widely used pharmacon increasing the resistance of the hydroxyapatite component of teeth to the acidic challenge (Hughes, J. et al., J Oral Rehabil 2004, 31, 4, 357-63). The compositions efficiently adhere to the surface of human dentin and can occlude dentinal tubules. In certain present embodiments, a surface modification with a stabilizing compound that can anchor to calcium, dentin or other tooth or gum material is provided. In some embodiments the stabilizing compound can be a phospholipid or a phosphoprotein. Phospholipids are well-known drug carriers (Singh, R., et al., Journal of Drug Delivery Science and Technology, Volume 39, 2017, Pages 166-179) and many are non- to low-toxic. Phosphoproteins can serve a similar purpose. By way of example described herein, a phosphoprotein such as casein is used to stabilize micro- and nanoparticles of calcium carbonate in aqueous suspensions, enabling their application in dentifrices. The novel stabilizing materials release fluoride under physiologically low pH regardless of the presence of other ingredients of saliva, sustaining the bulk fluoride concentration comparable with most fluorinated toothpastes. Low pH-triggered release selectively supplies the drug to the areas that need it the most, reducing the overall dose and ushering in a new type of targeted dentifrices. Active components can be released at a lower pH, which is characteristic for caries-affected tooth areas or elevated matrix metalloproteinase-9 (MMP-9) characteristic for inflamed gum tissue.

The compositions of the present invention enable targeted delivery of medications to the areas that need the medication the most. It reduces the patient's exposure to the medications, reduces side effects, and expands treatment options to take-home dentifrices. The particles instantly degrade to non-toxic compounds in the acidic environment of the stomach, which eliminates the issue of clearance from the body. Additionally, the microparticles of the present invention are safer than more commonly used nanoparticles, because the microparticles are too large to penetrate to the tooth pulp through the dentinal tubules.

The disclosure may practically be applied in desensitizing dentifrices and gum treatments with additional pharmaceutical effects depending on the drug cargo. For example, eugenol provides additional desensitizing and antibacterial action, and in the present invention can also be utilized as drug delivering material. Menthol provides a breath refreshing action, prednisolone provides an anti-inflammatory effect, and both can be utilized in the present invention as well.

In certain embodiments of the present invention, the compositions can occlude dentinal tubules, attach to the gum tissue and release a cargo drug to the oral cavity and to the pulp of the tooth, providing both desensitizing, antibacterial or any other effect depending on the drug cargo.

In another embodiment, the present invention is a dentifrice comprising a carrier material, wherein the carrier material is comprised of one or more of a casein-coated calcium carbonate microsphere, a casein-coated calcium carbonate nanosphere, a calcium carbonate/hydroxyapatite microplatelets, a calcium hydroxyapatite, or a mesoporous hydroxyapatite particle, and wherein the carrier material is used to encapsulate fluoride ions, eugenol or a pharmacon, and wherein the dentifrice has a pH between 6 and 8. The pharmacon can be eugenol, fluoride, doxycycline, chlorhexidine, prednisolone, or VTD. In a further embodiment, the casein is derived from skim milk. In a further embodiment, the dentifrice is an active ingredient in a toothpaste, a coating on dental floss, a toothpick, or a coating on a dental device.

### Example 1

The following Example describes the manufacture and preparation of the coating particles, and methods of use.

Calcium acetate, calcium carbonate, calcium chloride, casein, disodium hydrogen phosphate, eugenol, absolute ethanol, and polyethylene glycol methyl ether MW=550 were purchased from Sigma-Aldrich. Polyethylene glycol (PEG-300), potassium chloride, sodium bicarbonate, sodium dihydrogen phosphate, phosphoric acid, sodium fluoride, sodium chloride, sodium hydroxide, and Pierce™ BCA Protein Assay Kit were purchased from Thermo Fisher Scientific. Biotene® dry mouth oral rinse (artificial saliva) was purchased from GSK. All the reagents were analytical grade.

The Scanning Electron Microscopeused is a Zeiss Sigma Field Emission Scanning Electron Microscope (SEM), accelerating voltage of 4 kV (Carl Zeiss Microscopy, LLC, White Plains, NY, USA). The Powder X-ray diffraction was performed with a Rigaku Ultima IV XRD Diffractometer (Rigaku Analytical Devices, Wilmington, MA, USA), Cu Ka radiation (λ=1.540 A, 40 kV, 44 mA). The FTIR spectrometer is an IFS Equinox 55 Spectrometer, Bruker. The UV-Vis Spectrophotometer is a Varian Cary 50 UV-Vis Spectrophotometer, Agilent. The Fluoride meter is an ExStick FL700, Extech.

Preparation and Stabilization of Calcium Carbonate Microspheres. Calcium acetate-based solution was prepared-CaCO₃ (10 g, 0.1 mol), 50 ml of water, and acetic acid (12 g, 0.2 mol) were slowly mixed to control foaming. The mixture was diluted with water to 100 mL, kept overnight, and gravity filtered. A 10 mL portion of 0.3 M NaHCO₃ was added dropwise over 2 min to a vigorously stirred above-described calcium acetate-based solution (0.3M, 10 mL). The mixture was kept with no stirring for 5 min, then was stirred gently for 1 h, centrifuged at 8,000 rpm for 5 min, washed with water (3 times by 2 mL, centrifuged at 8,000 rpm for 5 min after each wash), redispersed in 1 ml of water, and dried on air. The yield was 100 mg. The particles were conjugated with casein (see below for casein conjugation).

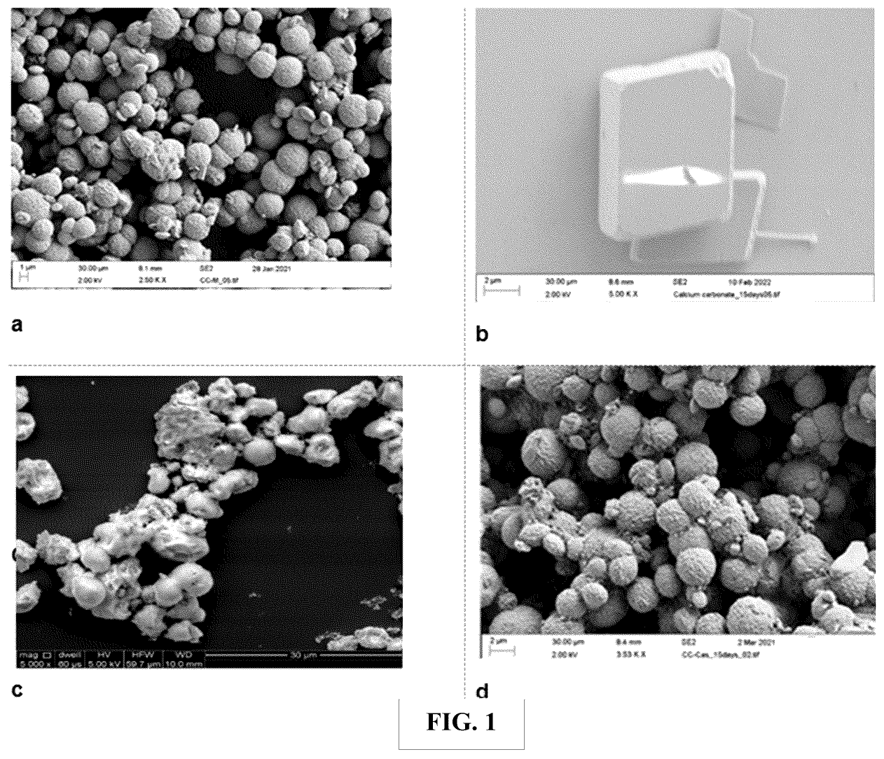

The SEM imaging of the 1-4 μm calcium carbonate microspheres prepared by the procedure shows their high porosity. The particles not stable in a 2 mg/ml aqueous suspension for more than 2 days were stabilized by pH 7.4 phosphate buffer saline (PBS) and much more efficiently—by a casein shell (7.2 μg casein/1 mg particles) (FIG. 1).

The calcium carbonate microspheres composed of thermodynamically unstable vaterite are known for their low stability in water because of their rearrangement to the more stable calcite (See Boyjoo, Y. et al). Reproducible synthesis of stable in the aqueous environment calcium carbonate particles of controlled morphology remains a challenge, which restricts a variety of their applications such as carriers of biomolecules (Sukhorukov, G. et al., J. Mater. Chem 2004, 14, 2073-81) and templates for microcapsules (Volodkin, D. et al., Langmuir 2004, 20, 3398-3406). Described in the present invention is a reproducible procedure for the precipitation of calcium carbonate microspheres from aqueous solutions of NaHCO₃ and a calcium acetate-based solution prepared by reacting CaCO₃ and aqueous acetic acid at the 1:2 molar ratio and removing the unreacted CaCO₃ by filtration. The pH value of the reaction mixture and evolving CO₂ provide ideal nucleation-crystal growth conditions for the calcium carbonate microspheres.

Since the unstable vaterite rearranges to calcite through the process of Oswald ripening, that requires the diffusion of calcium ions into the solution, the stability of the vaterite particles can be improved by suppressing the diffusion. While the as-prepared particles (FIG. 1a) are completely degraded after 2 days in water (FIG. 1b), most of them retained their morphology even after 15 days in pH 7.4 PBS (FIG. 1c) due to the formation of less soluble calcium phosphate on the surface of particles. Coating the particle with casein bound to the surface by its phosphoserine residues stabilizes the particles so efficiently that they remain intact even after 15 days in water (FIG. 1d). This may also be due to a scaffolding effect of the casein coat.

Preparation and Stabilization of Calcium Carbonate Nanospheres. For Solution A, polyethylene glycol methyl ether (Mw=550, 20 mL) was added to 5 ml of 1 M aqueous calcium acetate. For Solution B, polyethylene glycol methyl ether (Mw=550, 20 mL) was added to 5 ml of 1 M aqueous sodium bicarbonate. Calcium carbonate nanospheres were prepared according to the first step of the procedure described in Yang et al., describing the fabrication of hollow hydroxyapatite nanoparticles by acidic etching of core-shell calcium carbonate-hydroxyapatite nanoparticles (Yang, Y. et al., J Mater Chem B 2013, 1, 2447-50). Solution A and Solution B were mixed at room temperature without stirring and incubated for 10 s. The resulting cloudy mixture was vigorously stirred for 4 h at room temperature and centrifuged at 11,000 rpm for 10 min. The separated white particles were washed with nanopure water (2×40 mL), centrifuged at 11,000 rpm for 10 min after each washing, and dried on air. The yield was 0.1 g.

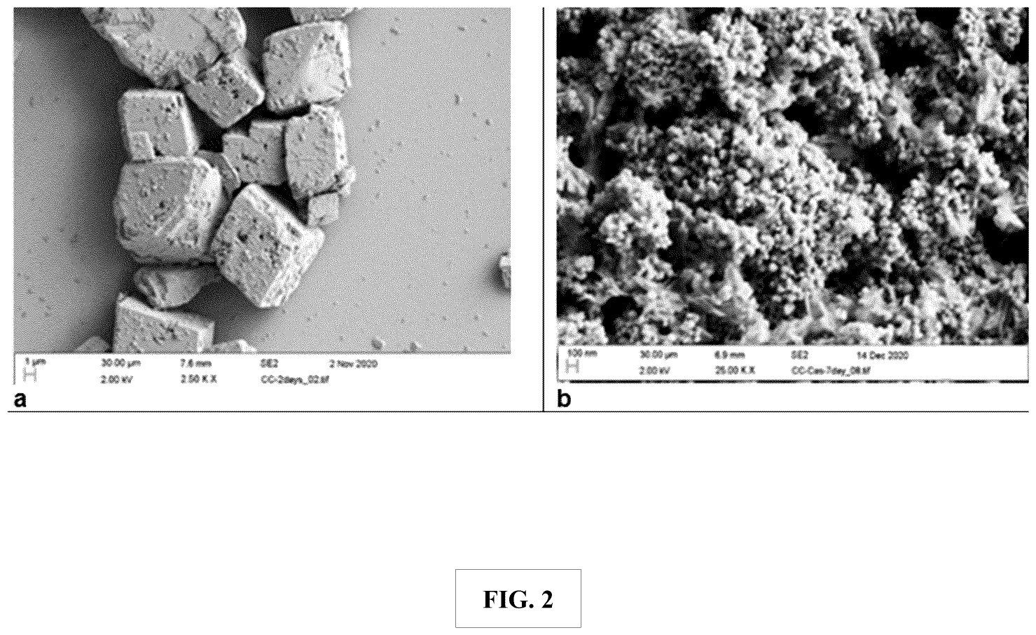

The SEM imaging of the 50-100 nm calcium carbonate nanospheres prepared by the procedure shows their tendency to aggregate. Similarly to their microspherical counterpart, the particles are not stable in a 2 mg/mL aqueous suspension and were efficiently stabilized by a casein shell (5.6 μg casein/1 mg particles). (FIG. 2).

The stabilizing effect of casein on calcium carbonate spherical particles was found to be equally effective for both nanoparticles (50-100 nm) synthesized by a polymer-assisted method and microparticles (1-4 μm) (FIG. 1,2). While the calcium carbonate nanospheres completely degraded in an aqueous 2 mg/ml suspension for 7 days (FIG. 2a), their casein-coated modification completely retained its morphology (FIG. 2b). Both types of particles are stable as a dry powder even without casein, which confirms the role of the calcium ions diffusion in the vaterite rearrangement.

Preparation and Stabilization of Calcium Carbonate/Hydroxyapatite Microplatelets. For Solution A, polyethylene glycol methyl ether (Mw=300, 20 mL) was added to 5 ml of 1 M aqueous calcium acetate. For Solution B, polyethylene glycol methyl ether (20 mL) was added to 5 ml of 1 M aqueous sodium bicarbonate. Solution A and Solution B were mixed at room temperature without stirring and incubated for 10 s. The resulting cloudy mixture was vigorously stirred (1,200 rpm) with a magnetic stir bar for 4 h at room temperature. Next, the suspension was vigorously stirred (1,200 rpm) with 2 mL of 0.1 M phosphoric acid for 17 h and centrifuged (11,000 rpm, 10 min). The separated white particles were washed with nanopure water (2×40 mL), centrifuged (11,000 rpm, 10 min) after each washing step, and dried on air. The yield was 0.241 g.

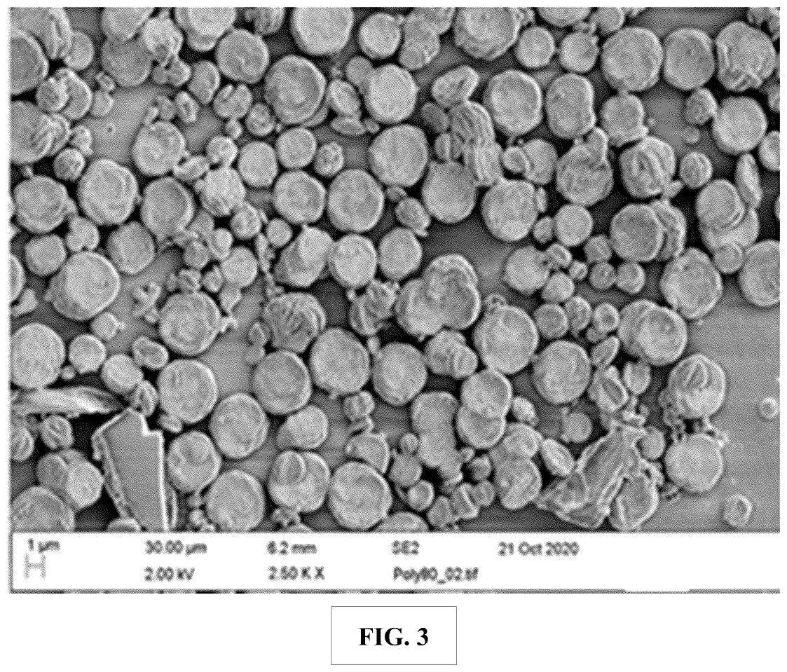

The SEM imaging of calcium carbonate/hydroxyapatite core-shell microparticles prepared by our procedure reveals their morphology as 1-3 μm diameter platelets (FIG. 3). The particles were stable in a 2 mg/mL aqueous suspension for at least 15 days.

To enhance the stabilizing effect of a phosphate layer on calcium carbonate particles, calcium carbonate/hydroxyapatite core-shell microparticles were prepared by treating intermediate calcium carbonate particles with aqueous phosphoric acid. The 1-4 μm particles did not undergo any visible changes after 15 days of exposure to water (FIG. 3). Therefore, the hydroxyapatite shell coating prevented the rearrangement of calcium carbonate to the cubic calcite and produced platelet-like particles rather than spherical ones.

Preparation and Stabilization of Hydroxyapatite Particles. The particles were prepared according to Huang et al. (Mater Sci 2007, 42, 8599-8605). Briefly, a 100 mL portion of aqueous 0.05 M CaCl₂) with PEG-300 (1.5% w/v), kept at room temperature for 12 h, was added with a burette at the flow rate of 1.6 mL/min into 100 mL of aqueous 0.03 M Na₂HPO₄ with constant stirring at 1,000 rpm. The mixture was kept for 48 h at room temperature in a sealed vial and centrifuged at 11,000 rpm for 8 min. The product was washed with distilled water and ethanol three times, dried at 60° C. for 16 h, and calcined at 500° C. for 2 h. The yield was 0.26 g.

Preparation and Stabilization of Mesoporous Hydroxyapatite Particles. The particles were synthesized based on Wu et al. (Curr Nanosci 2011, 7, 6, 926-31). Briefly, polyethylene glycol methyl ether (20 mL) was added to 5 ml of 1 M aqueous calcium acetate to prepare Solution A. 20 ml of polyethylene glycol methyl ether was added to 5 ml of 1 M aqueous sodium bicarbonate to prepare Solution B. Solution A and Solution B were mixed at room temperature without stirring and were incubated for 10 seconds. The resulting cloudy mixture was stirred (1000 RPM) for 4 h at room temperature. The suspension was stirred (1000 RPM) with 2 mL of 0.01 M of phosphoric acid for 17 h and centrifuged (8000 rpm, 10 min). After each step, the separated white particles were washed with nanopure water (3×20 mL) and centrifuged (8000 rpm, 10 min). The precipitate was suspended in 10 ml of sodium acetate buffer (pH˜6.0, 0.025 M) and stirred for 30 min. The particles were separated by centrifugation (8000 rpm, 10 min), washed three times with 20 ml of nanopure water, and dried overnight at ambient temperature to afford the final product. Yield 0.85 g.

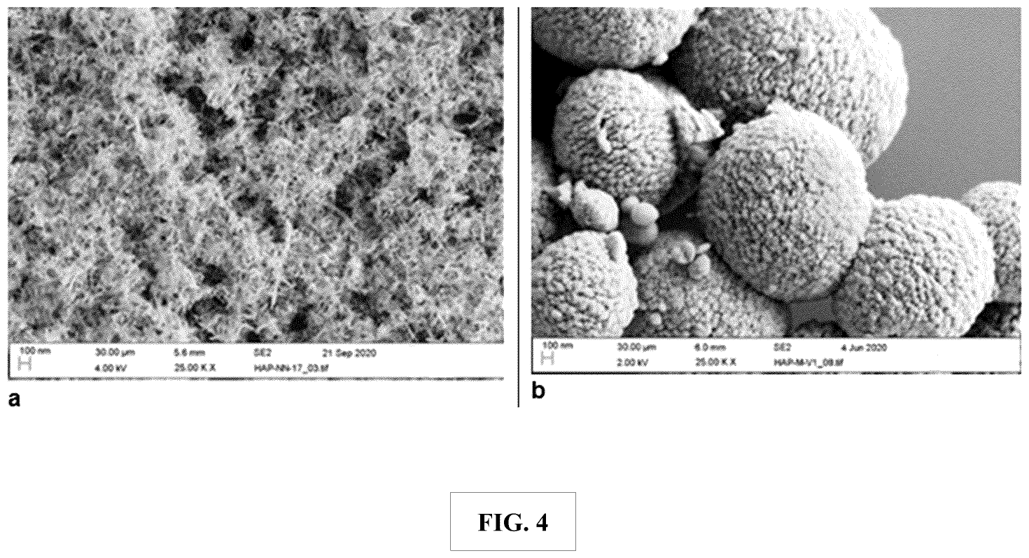

Hydroxyapatite nanoneedles (see Huang, F et al.) and mesoporous microparticles (See Wu, K et al.) were synthesized according to known procedures. The particles' size and morphology confirmed by Scanning Electron Microscopy (SEM) imaging (FIG. 4) were consistent with those reported in the literature (Huang, F et al., Wu, K et al.). The X-Ray Diffraction (XRD) pattern of the hydroxyapatite nanoneedles (2 T 26, 29, 32-34, 40, 46-54) was consistent with the presence of the (002), (211), (300), (202), (310), (222), (213) and (411) reflection planes of the hydroxyapatite phase according to the JCPDS files (Hong, J. et al., Chem. Mater 2006, 18, 21, 5111-8).

The hydroxyapatite particles of two distinctly different morphologies (nanoneedles and mesoporous spherical microparticles) were prepared to explore if the pH-dependent drug delivery functionality of the particles and their conjugates with casein can be tuned by their morphology. The needle-like particles were expected to better adhere to dentin and release the drug faster, while their mesoporous spherical counterparts were expected to hold larger amounts of the drug.

Conjugation of Particles with Casein. A 1 mL portion of aqueous 1 mg/mL casein was added to 5 mg of particles, and the mixture was mixed on a vortex for 5 min, placed in a Roto-Mini rotating mixer with 24 RPM for 30 min, and centrifuged at 8,000 for 30 min. The supernatant was analyzed by the BCA protein assay as described by Walker, J, to determine the amount of casein not conjugated with the particles (Walker, J. The bicinchoninic acid (BCA) assay for protein quantitation. In The protein protocols handbook, Springer: New York, 2009, 11-5). The amount of casein conjugated to each type of particles is presented in Table 1.

Human teeth were donated through extraction as a medically necessary procedure at the patients' consent. The procedure was not performed with the purpose of performing any research.

For the preparation of dentin, tooth samples were collected, prepared, and cleaned by the previously reported method in Rashwan, K. et al. The enamel layer of the tooth specimens was removed to aid in sample processing, except for one sample where the enamel was intentionally preserved for comparison. A tooth was mounted on a Beuhler Isomet Slow-speed saw, sectioned into 2.5 mm×2.5 mm×0.5 mm mid-coronal dentin slices with a diamond blade, rinsed with nanopure water, and ultrasonicated for 1 min in 0.2 N citric acid to remove saw smears and debris. The presence of open dentinal tubules in the dentin samples was confirmed by SEM imaging.

For the application of particles to dentin, a slice of dentin was gently brushed (1 min from each side) with a paste of 100 mg of particles and 1 ml of water. Each side of the dentin was rinsed with water for 10 s, and the slice was sonicated in water for 30 s. The samples were rinsed with water for 10 s for each side and kept on the air.

For the loading of particles with eugenol in a suspension, a 5 mg portion of particles and 1.0 ml of 0.2 mg/mL eugenol in PBS buffer pH 7.4 was mixed on a vortex for 5 min, placed in a Roto-Mini rotating mixer with 24 RPM for 24 h, and centrifuged at 8000 rpm for 6 min. The supernatant A was collected. The precipitate was mixed with 0.5 mL of PBS buffer pH 7.4, vortexed for 2 min, and centrifuged at 8000 rpm for 6 min. The supernatant B was mixed with supernatant A, and the concentration of unbound eugenol was determined by absorbance at 280 nm.

For the loading of eugenol on dentin or enamel slices, a dentin or enamel slice was placed in 15 mL of the eugenol solutions (0.1 g/mL in PBS buffer, pH 7.4). The solution was gently magnetically stirred (250 rpm) for 24 h at room temperature. The drug-loaded specimen was collected with a spatula and washed with PBS buffer (pH 7.4) two times to remove the surface adsorbed eugenol.

For the loading of particles with fluoride in a suspension of casein-coated calcium carbonate microspheres, a 10 mg portion of particles and 1.0 mL of 1 mg/mL or 1000 ppm fluoride solution in nanopure water was mixed on a vortex for 5 min, placed in a Roto-Mini rotating mixer with 24 RPM for 2 h and centrifuged at 8,000 rpm for 6 min. The supernatant A was collected. The precipitate was mixed with 1 mL of nanopure water, vortexed for 2 min, and centrifuged at 8000 rpm for 6 min. Then, supernatant B was collected. After that, supernatant A and B were mixed and diluted to 20 ml with nanopure water. Again, 1 mL of the diluted solution was taken and diluted to 20 mL with nanopure water. Then the solution was read by a fluoride meter, and amount of the unbounded fluoride was calculated according to equations 1-2. Then fluoride-loaded particles were conjugated with casein according to the procedures described above.

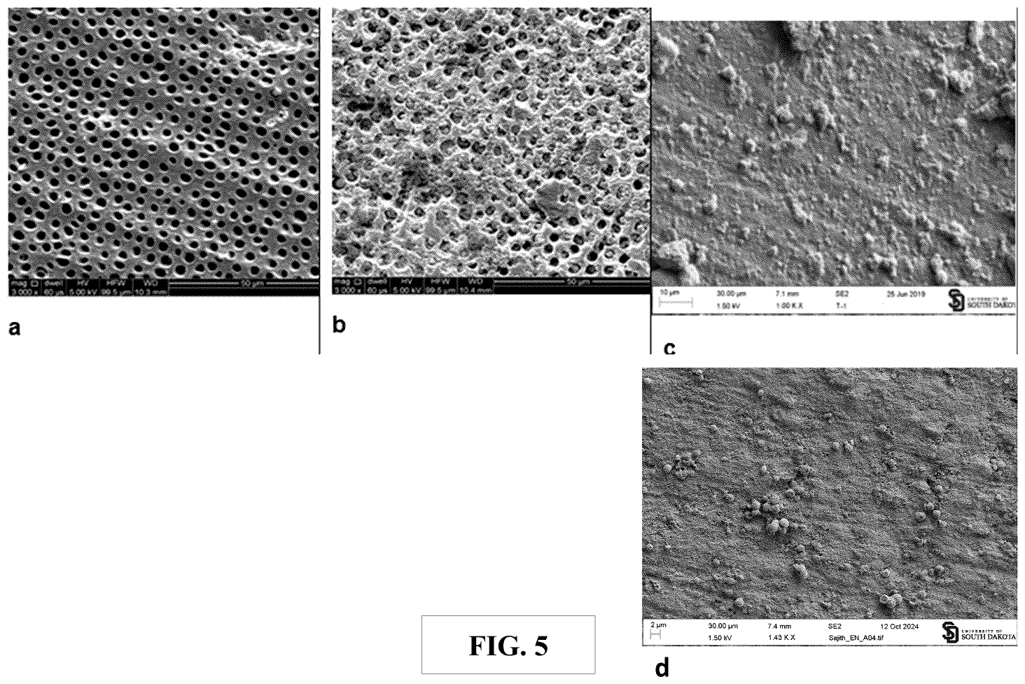

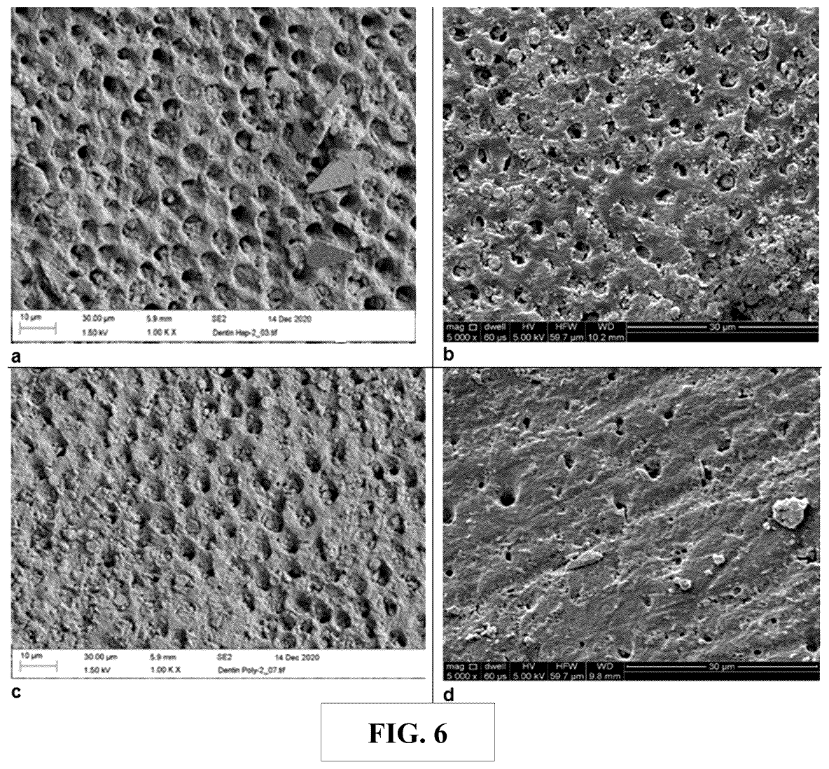

Adhesion of Particles to Dentin or Enamel. All synthesized particles have shown their ability to occlude dentinal tubules after application of an aqueous (100 mg/mL) paste followed by 1 min ultrasonication in water (FIG. 5-6). To qualitatively evaluate the capability of particles to adhere to dentin and occlude its tubules by SEM imaging, it is critical to make sure that the dentinal tubules are open before applying particles (FIG. 5a). This is achieved by removing any saw smears or debris from the surface of dentin by sonication with an aqueous citric acid followed by thorough rinsing with water. After the application of particles, all samples were sonicated in water for 30 s to remove loosely associated particles from dentin. The casein-stabilized calcium carbonate microspheres efficiently adhere to dentin and occlude its tubules (FIG. 5b). The most efficient visible occlusion was observed for mesoporous hydroxyapatite microparticles (FIG. 5c), perhaps due to the mechanically rough surface of particles (FIG. 4b). For hydroxyapatite nanoneedles and calcium carbonate/hydroxyapatite microplatelets, the casein coating does not apparently affect their occlusive properties (FIG. 6).

Loading and Release of Eugenol by Aqueous Suspensions of Particles. Table 1 summarizes the capacity of bare and casein-coated particles for eugenol.

TABLE 1
The amount of casein conjugated with calcium
carbonate/hydroxyapatite-based particles.

| Particle Type | Casein Conjugation Capacity |
|---|---|
| Calcium carbonate microsphere | 7.2 μg ± 1.1 μg |
| | eugenol/1 mg particles |
| Calcium carbonate nanosphere | 5.6 μg ± 1.3 μg |
| | eugenol/1 mg particles |
| Hydroxyapatite nanoneedles | 5.8 μg ± 1.1 μg |
| | eugenol/1 mg particles |
| Calcium Carbonate/Hydroxyapatite | 5.0 μg ± 1.2 μg |
| Microplatelets | eugenol/1 mg particles |

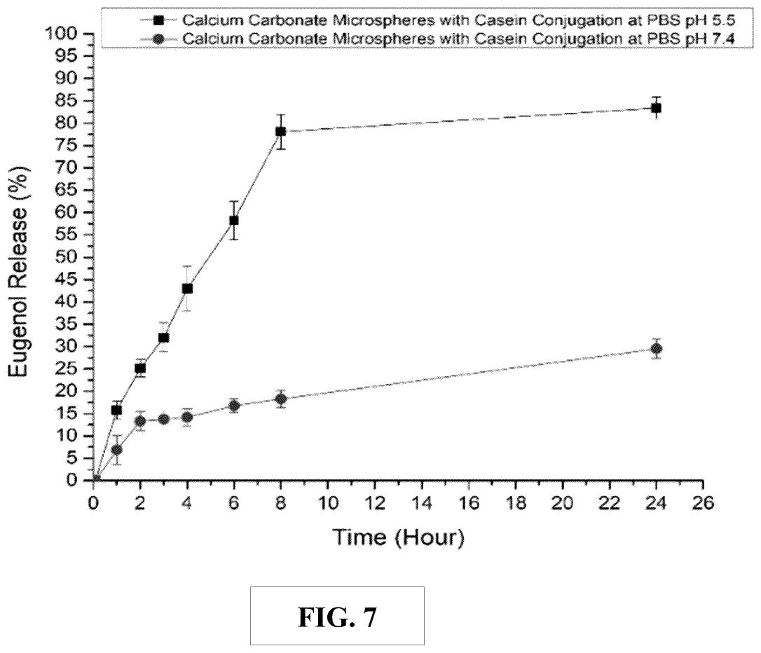

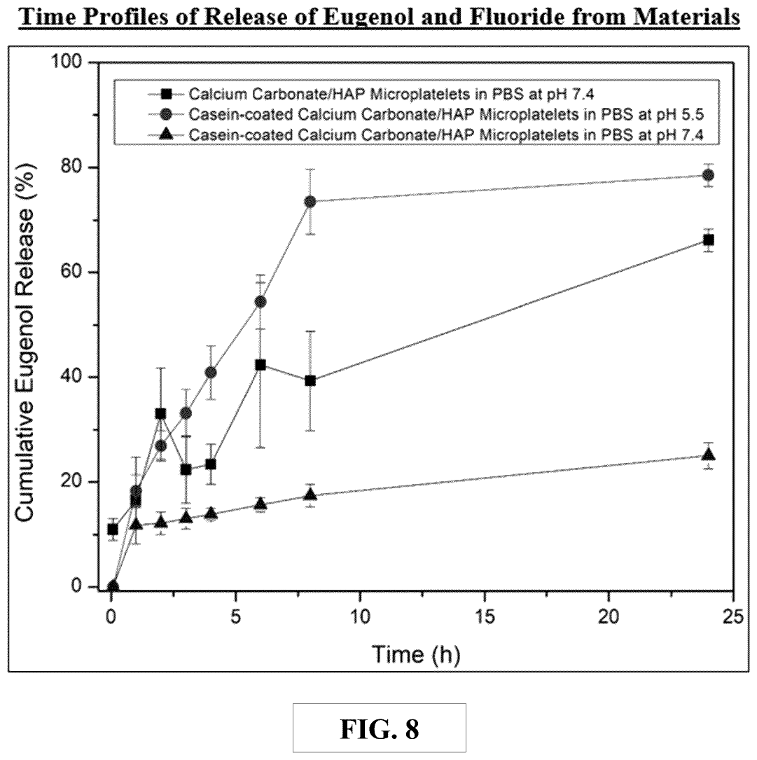

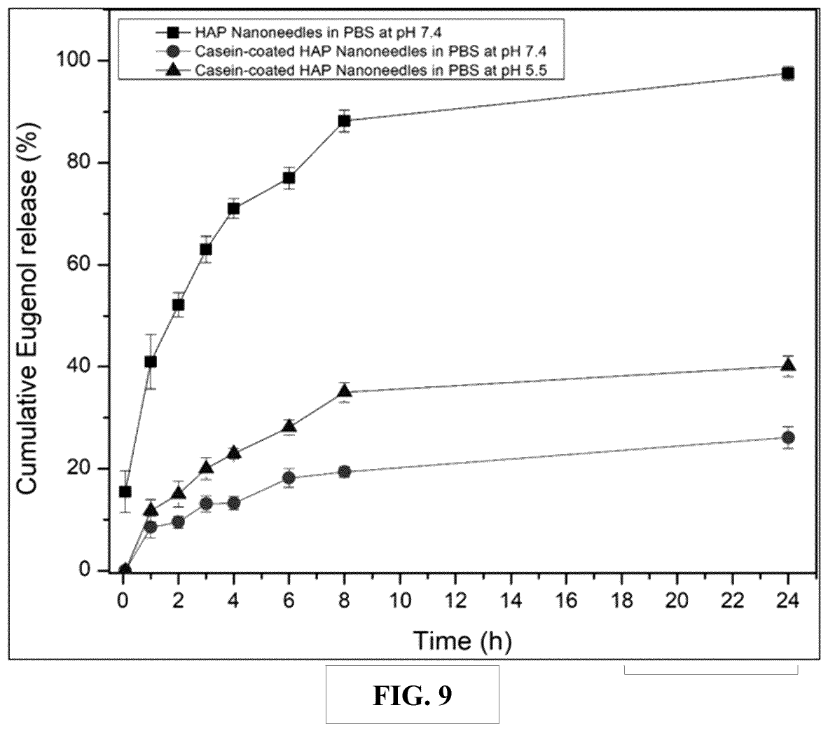

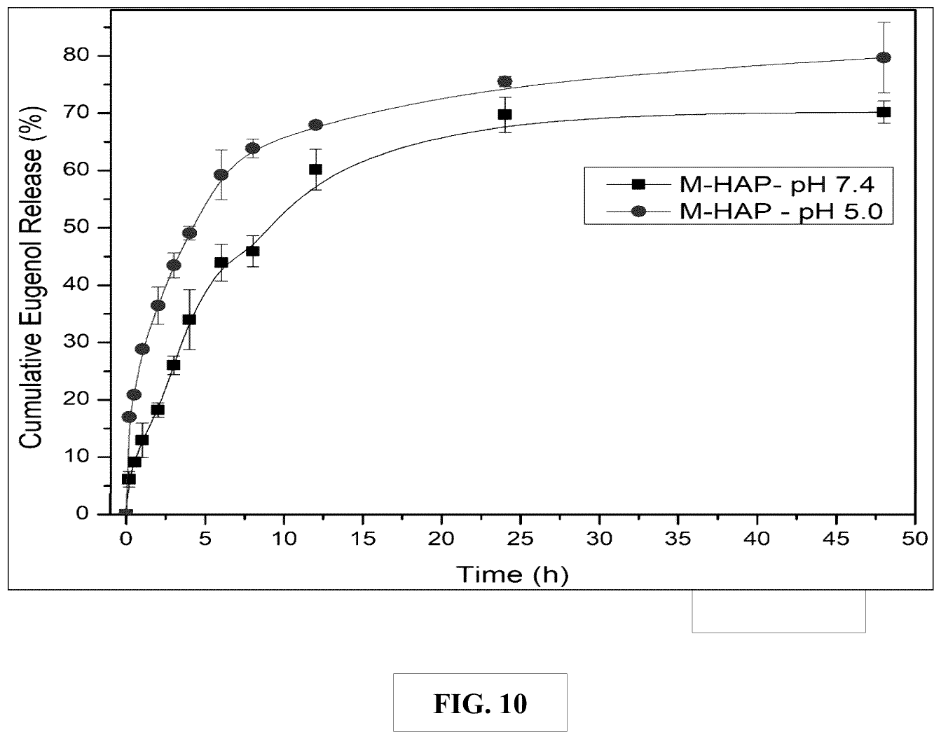

The time profiles for the release of eugenol by casein-coated calcium carbonate microspheres measured for the pH values of 7.4 and 5.5 demonstrated the acid-triggered drug release. (FIG. 7). The time profile for the release of eugenol by calcium carbonate/hydroxyapatite microplatelets at pH 7.4 was consistently erratic (FIG. 8, square line). Coating particles with casein smoothens and slows the release of eugenol (FIG. 8, triangle line). The circle line in FIG. 8 corresponds to the release of eugenol from casein-coated calcium carbonate/hydroxyapatite microplatelets. The time profiles for the release of eugenol by hydroxyapatite nanoneedles at pH 7.4 (FIG. 9, square line), casein-coated hydroxyapatite nanoneedles at pH 7.4 (FIG. 9, circle line), and pH 5.5 (FIG. 9, triangle). The time profiles for the release of eugenol by mesoporous hydroxyapatite microparticles at pH 7.4 are shown in FIG. 10 (square line) and at pH 5.5 (circle line).

As shown in Table 1, the morphology of particles does not substantially affect their capacity for eugenol, which tends to be about 15% higher for hydroxyapatite-core particles than for calcium carbonate-core particles.

The casein-coated calcium carbonate microspheres slowly release eugenol into the solution at pH 7.4. Only 25% of the loaded eugenol was released after 24 h (FIG. 7, circle line). In contrast, at pH 5.5, 80% of eugenol was released after 8 h, sustaining the concentration of eugenol 67.2 μg/mL. This estimate of the maximum attainable concentration of the released eugenol is close to the concentration at which eugenol inhibits the proliferation of cancerous cells (82 μg/mL) (Jaganathan, S. et al., Molecules 2012, 17, 6290-6304) and is well below the concentration at which it starts to show toxicity for mammalian cells at prolonged exposures and bactericidal activity (164 μg/mL) (Da Silva, F. et al., Synthesis, characterization, and evaluation of antibacterial and antioxidant activities. Chem Cent J 2018, 12, 34-42). While the therapeutical application of eugenol itself is limited by the unfavorable balance of its therapeutical activity and toxicity (Markowitz, K. et al., Oral Surg Oral Med Oral Pathol 1992, 73, 729-37), the local concentrations of eugenol in a confined environment (inside dentinal tubules or underneath biofilms) can be expected to exceed these levels at least for a brief time, which underlines the importance of targeted delivery of eugenol by novel materials. Only 15% of eugenol was released from the particles after 8 h at pH 7.4. About 5× increase of the drug release triggered by acid is crucial for the targeted delivery of eugenol to the areas of teeth exposed to the bacteriogenic acids starting the process of tooth decay.

The calcium carbonate/hydroxyapatite core-shell microplatelets have shown a similar capacity for eugenol as casein-coated calcium carbonate microspheres. Interestingly, the non-coated microplatelets released eugenol erratically (FIG. 9, square line), probably, due to the heterogeneous nature of their surface and re-adsorption of eugenol from the solution. The casein coating sealed eugenol inside the particles, slowed down (0.3× after 8 h), and smoothened its release (FIG. 8, triangle line). The casein-coated core-shell microplatelets were as responsive to the acid-triggered release (5× increase after 8 h) as calcium carbonate microspheres.

Switching the hydroxyapatite morphology from microplatelets to nanoneedles increases the particles' capacity for eugenol by ˜30%, but decreases their ability to hold eugenol, probably, because of the prevalence of its surface adsorption versus diffusion inside the particles. After 8 h, 90% of eugenol was released (FIG. 9, square line). Similarly, to other types of particles, coating with casein substantially increased the retention of eugenol by the particles: only 15% was released after 8 h, and 75% of eugenol remained inside the particles even after 24 h (FIG. 9, circle line). However, acidification to pH 5.5 exerted just a moderate 1.7× increase in the release after 8 h (FIG. 9, triangle line). Therefore, casein-coated hydroxyapatite nanoneedles hold more eugenol than calcium carbonate-based microspheres and microplatelets for 24 h, but they are much less sensitive to the acid-triggered release.

As expected, mesoporous hydroxyapatite particles were able to hold eugenol better (45% of eugenol released after 8 h, FIG. 10) than hydroxyapatite nanoneedles (90% of eugenol released after 8 h, FIG. 10). Similarly, to hydroxyapatite nanoneedles, mesoporous hydroxyapatite particles have shown moderate sensitivity to the acid-triggered release (1.4× increase of the release upon switching pH from 7.4 to 5.5, FIG. 10). The observed tendency of calcium carbonate-based particles to be more sensitive to the acid-triggered drug release than hydroxyapatite particles is not surprising considering the higher solubility of calcium carbonate than hydroxyapatite at pH 5.5.

Coating with casein played a unique role for each type of particles. The casein layer stabilized the calcium carbonate microspheres, smoothened the release of eugenol by calcium carbonate/hydroxyapatite microplatelets, enabled the retention of eugenol by hydroxyapatite nanoneedles, and was not needed for mesoporous hydroxyapatite particles. In addition, casein is digested by the MMP-9 enzyme overexpressed by inflamed tissues with leads to the preferential drug release at the inflamed tissues.

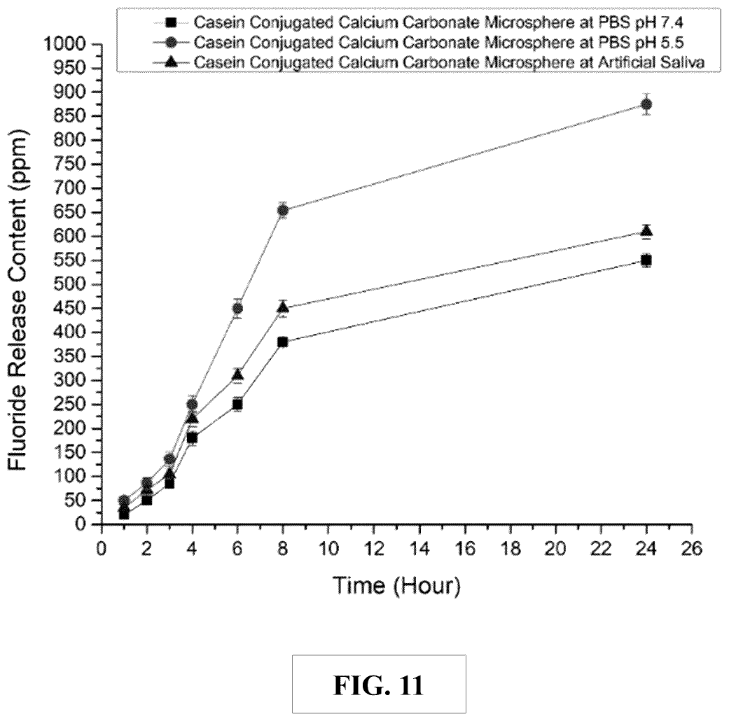

Release of Fluoride by Casein-Coated Calcium Carbonate Microspheres. The time profiles for the release of fluoride by the particles are in FIG. 11. The casein-coated calcium carbonate microspheres are at pH 7.4 (FIG. 11, square line), casein-coated calcium carbonate microspheres are at pH 5.5 (FIG. 11, circle line), and artificial saliva are at pH 6.9 (FIG. 11, triangle line).

The casein-coated calcium carbonate microspheres as a candidate for the acid-triggered release of eugenol in dentifrice applications were explored for their ability to release the fluoride-ion as well. The fluoride release time profiles were collected at three values of pH: 7.4 (Phosphate Buffered Saline (PBS) buffer), 6.9 (artificial saliva Biotene®), and 5.5 (PBS buffer). FIG. 11 shows a clear trend of the substantially accelerated release of eugenol as the pH value decreases, sustaining the maximum cumulative fluoride concentration of 850 ppm, which is comparable with the 1,000-1,500 ppm for most fluorinated toothpaste. This trend is not affected by the ingredients of artificial saliva, which speaks to the value of the particles for dentifrice applications as a source of both eugenol and fluoride.

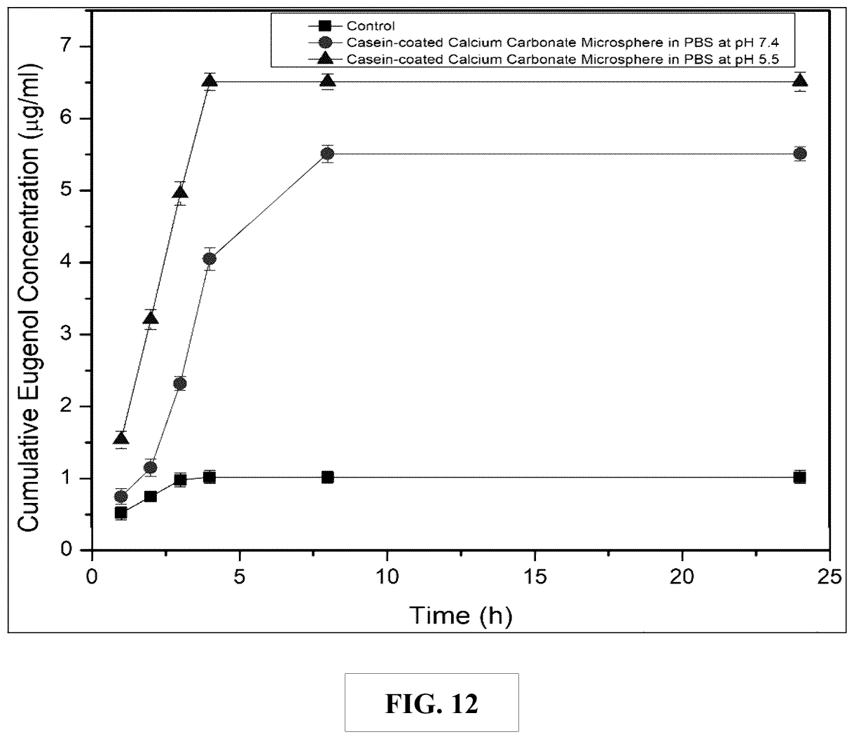

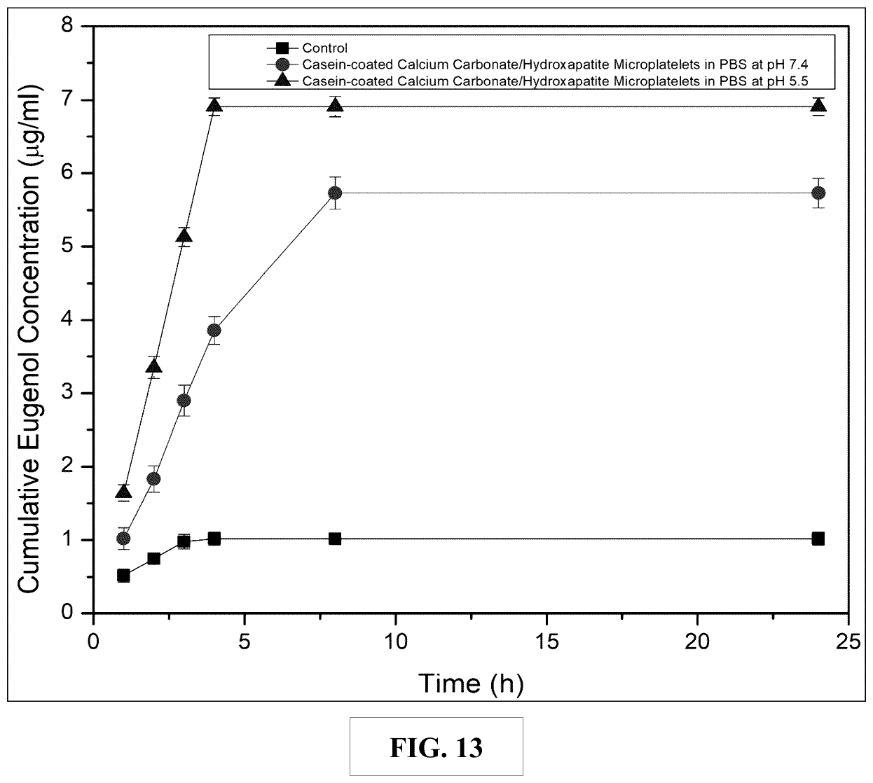

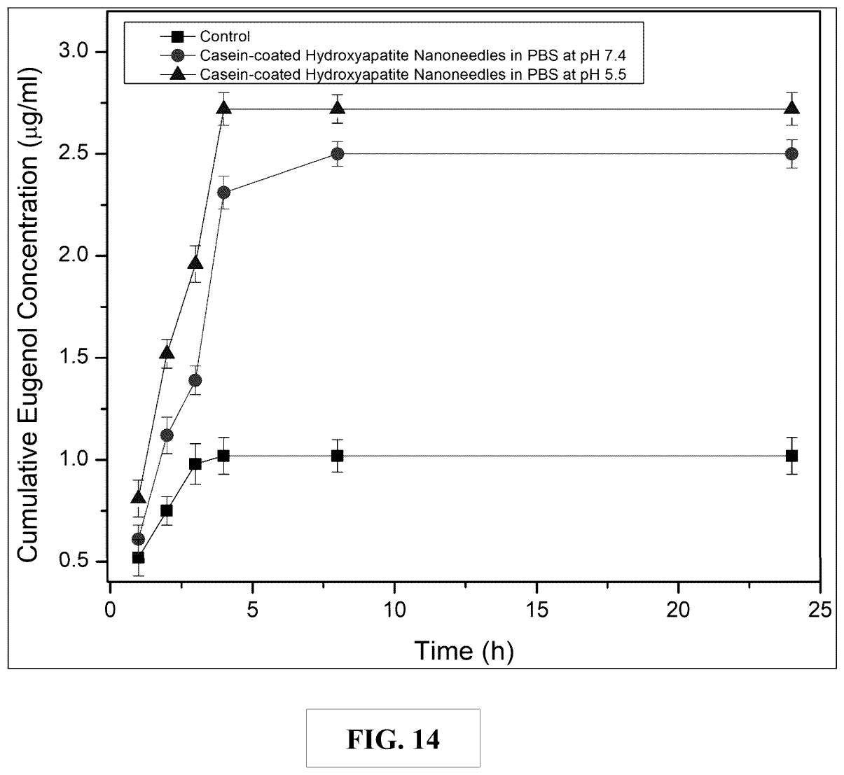

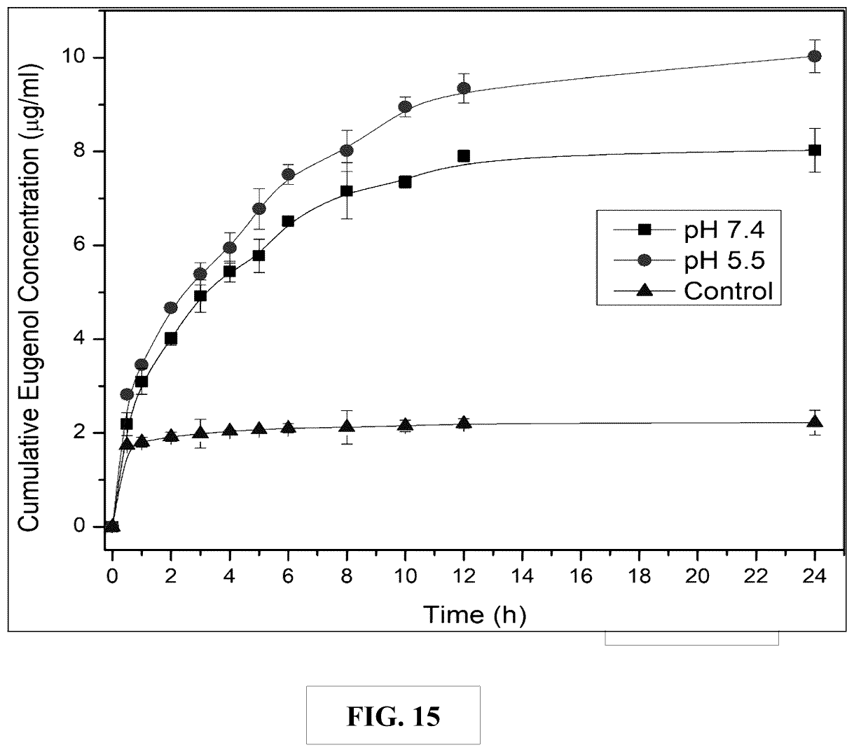

Release of Eugenol by Particles adhered to dentin. FIG. 12 shows the time profiles for the release of eugenol by dentin treated with casein-coated calcium carbonate microspheres at pH 7.4 (FIG. 12, square line) and pH 5.5 (FIG. 12, circle line). Release from untreated dentin is shown as FIG. 12, triangle line. The time profiles for the release of eugenol by dentin treated with casein-coated calcium carbonate/hydroxyapatite microplatelets at pH 7.4 are shown in FIG. 13 (square line) and pH 5.5 FIG. 13, (circle line). Release from untreated dentin is shown in FIG. 13, triangle line. The time profiles for the release of eugenol by dentin treated with casein-coated hydroxyapatite nanoneedles at pH 7.4 are shown in FIG. 14, (square line) and pH 5.5 (circle line). Release from untreated dentin is shown in FIG. 14, triangle line. The time profiles for the release of eugenol by dentin treated with mesoporous hydroxyapatite particles at pH 7.4 is shown in FIG. 15, square line and pH 5.5 (circle line). Release from untreated dentin is shown in FIG. 15, triangle line.

While dentin itself can absorb eugenol, the treatment with eugenol-loaded particles increased its capacity X3 for casein-coated hydroxyapatite nanoneedles and X5-7 for other types of particles (FIG. 12-15). Similarly, to the aqueous suspensions, acidification from pH 7.4 to pH 5.5 roughly doubled the eugenol release in 2-4 h by casein-coated calcium carbonate microspheres and casein-coated calcium carbonate/hydroxyapatite microplatelets. For the particles with the hydroxyapatite core, the pH effect on the release was less pronounced than for the particles with the calcium carbonate core due to the higher basicity of calcium carbonate. The overall amount of eugenol released by hydroxyapatite-core particles at pH 7.4 was about 15% less than the amount released at pH 5.5. The bulk concentrations of eugenol released by the particles on dentin (5-10 μg/mL) are much lower than for eugenol released in an aqueous suspension and are well below the biologically active concentrations. Therefore, the particles hold eugenol at the surface of dentin and locally release it at lower pH values while eliminating the risk of systemic exposure to eugenol, including its ingestion. The evaluation of local concentrations of eugenol can potentially be performed by using a dentin-embedded microfluidic device (França, C. et al., Lab Chip 2020, 20, 405-13).

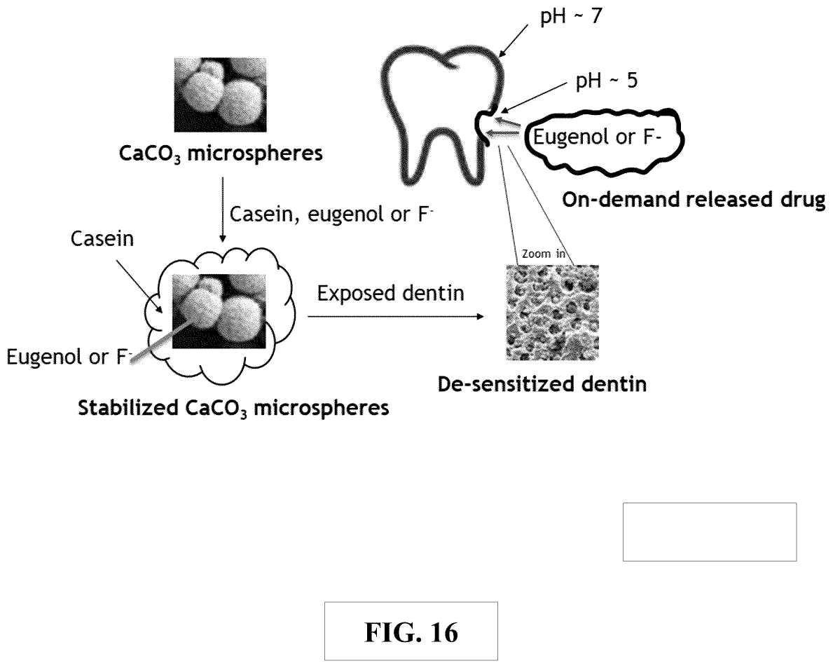

Conclusions. The surface modification with casein stabilizes micro- and nanoparticles of calcium carbonate in aqueous suspensions, and therefore enables their application in dentifrices (FIG. 16). A series of nano- and microparticles based on calcium carbonate, calcium hydroxyapatite, and casein hold an antibacterial, desensitizing, and anticancer eugenol, adhere to dentin, and release eugenol in an acid-triggered manner, sustaining the bulk eugenol concentration in a solution comparable with its anti-proliferation levels, but below the antibacterial and mammalian toxicity levels. The adhesion of particles to dentin enables the tooth surface to hold and release eugenol. The ability of particles to hold and release eugenol depends on their morphology and composition, with the casein-coated calcium carbonate microspheres being the most acid-sensitive and most promising for dentifrice applications. The casein-coated and fluoride-loaded calcium carbonate microspheres release fluoride in a pH-dependent manner regardless of the presence of other ingredients of the saliva, sustaining the bulk fluoride concentration comparable with most fluorinated toothpastes.

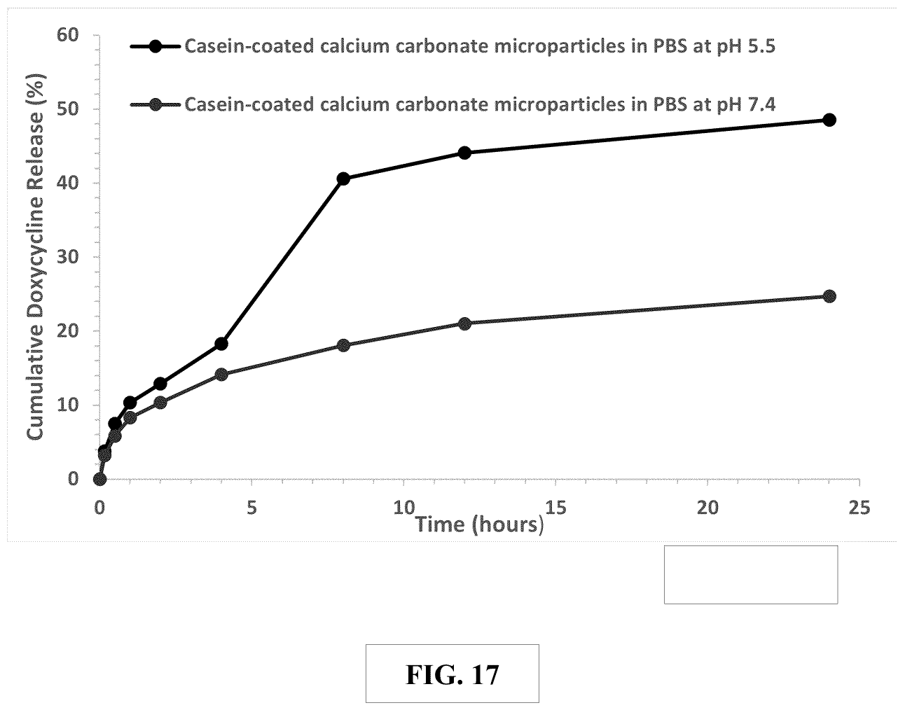

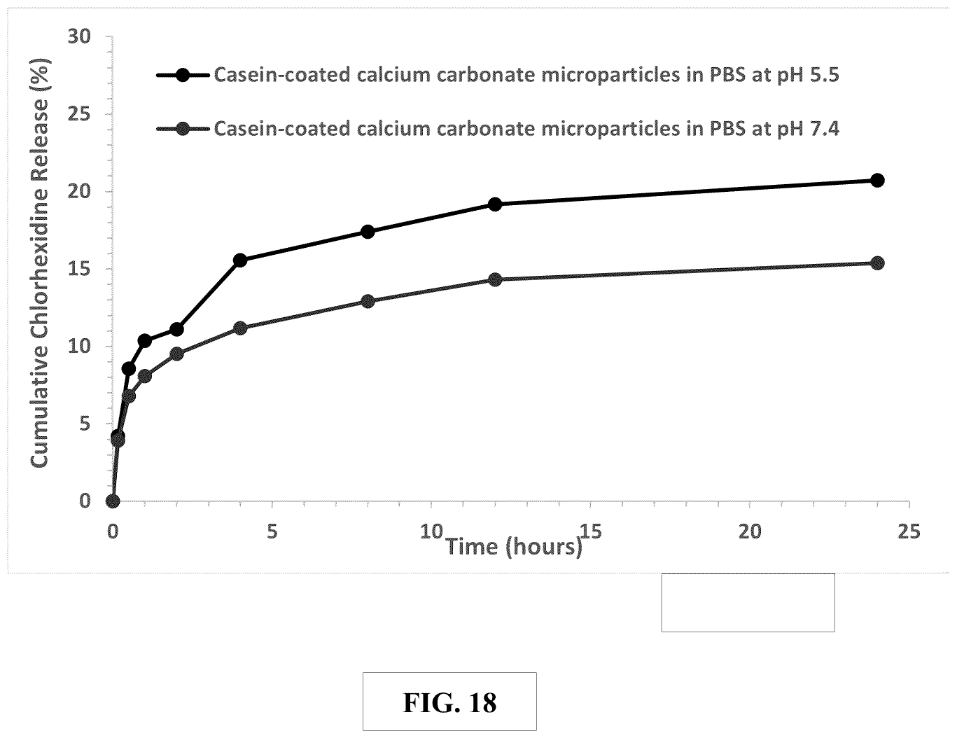

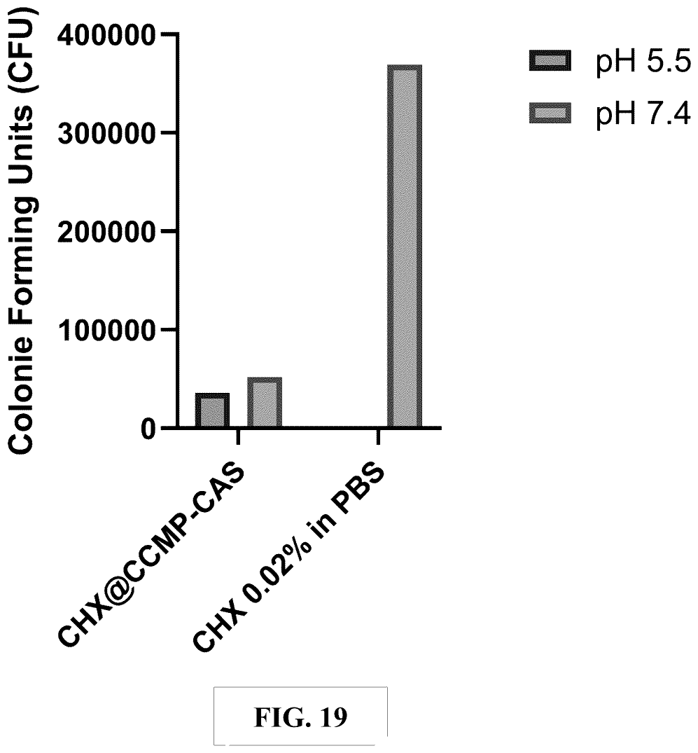

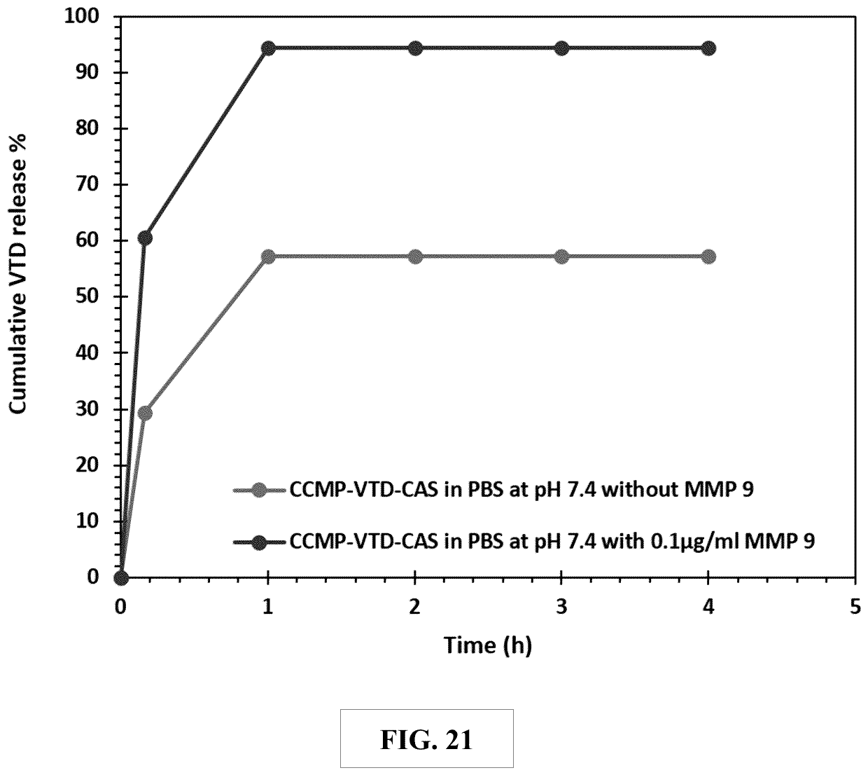

Microparticles of hydroxyapatite, calcium carbonate, or calcium citrate loaded with a drug such as eugenol, fluoride, doxycycline, chlorhexidine and prednisolone, or VTD are able to occlude dentinal tubules, attach to dentin or enamel, and release a cargo drug to the oral cavity and potentially to the pulp of the tooth, providing both desensitizing, antibacterial or any other effect depending on the drug cargo. See FIG. 17 for an example of the results at varying pH with doxycycline, FIG. 18 for chlorhexidine and FIG. 21 for VTD. Chlorhexidine-loaded and casein-coated calcium carbonate microparticles (CHX@CCMP-CAS) significantly suppress growth of colonies of Streptococcus mutan (caries-causing bacteria) comparing with the control (0.02% chlorhexidine) especially at pH 5.5 when the drug release is triggered (See FIG. 19). Coating of the particles with casein or another surface modifier stabilizes microparticles of calcium carbonate, improves adhesion of particles to the tooth or gum tissue and blocks the drug release. The drug release is triggered under acidic conditions characteristic for the presence of cariogenic bacteria, MMP-9 enzyme produced by inflamed tissues and contributing to the failure of dental implants. The particles can be administered as an active ingredient of a dentifrice including a coating of a functionalized dental floss or a dental device. Casein-coated calcium carbonate microspheres is an example of calcium carbonate microspheres stabilized by a non-toxic agent. The acid-triggered drug release is based on the disintegration of the particles by acids. MMP-9—and other MMP-enzymes-triggered drug release is based on digesting the gate-keeping casein by MMP-9.

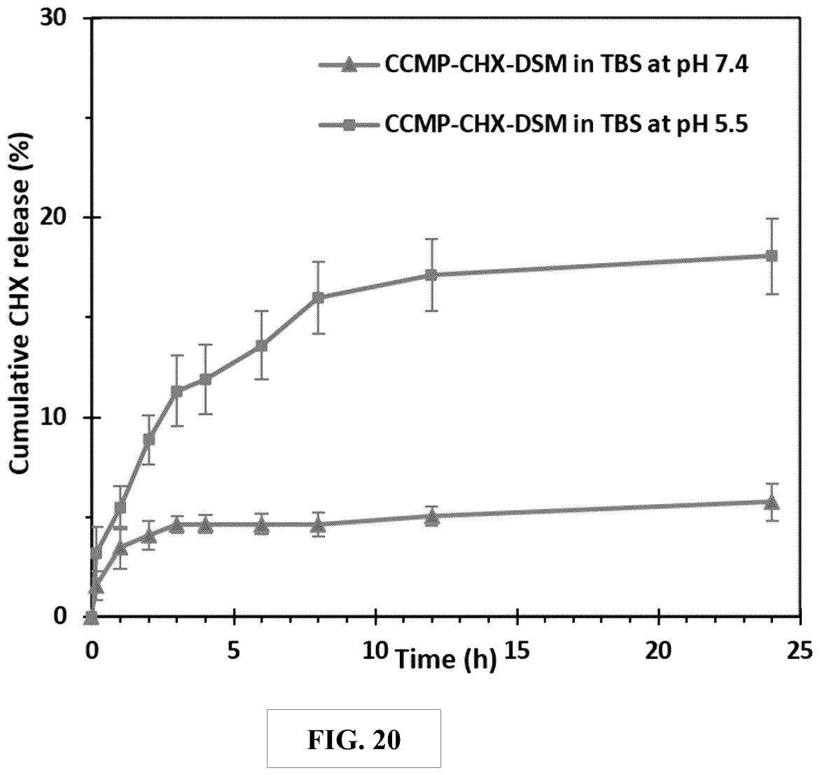

Alternative methods may be used to provide alternative coatings or encapsulation material. For example, FIG. 20 illustrates the results of using an alternative encapsulation using diluted skim milk that performs better for drug release than casein described above. Additionally, CaCO₃ microparticles can be prepared “top-down” through ball milling eggshells.

While the various systems described above are separate implementations, any of the individual components, mechanisms, or devices, and related features and functionality, within the various system embodiments described in detail above can be incorporated into any of the other system embodiments herein.

The terms “about” and “substantially,” as used herein, refers to variation that can occur (including in numerical quantity or structure), for example, through typical measuring techniques and equipment, with respect to any quantifiable variable, including, but not limited to, mass, volume, time, distance, wavelength, frequency, voltage, current, and electromagnetic field. Further, there is certain inadvertent error and variation in the real world that is likely through differences in the manufacture, source, or precision of the components used to make the various components or carry out the methods and the like. The terms “about” and “substantially” also encompass these variations. The term “about” and “substantially” can include any variation of 5% or 10%, or any amount-including any integer-between 0% and 10%. Further, whether or not modified by the term “about” or “substantially,” the claims include equivalents to the quantities or amounts.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range. Although the various embodiments have been described with reference to preferred implementations, persons skilled in the art will recognize that changes may be made in form and detail without departing from the spirit and scope thereof.

Although the various embodiments have been described with reference to preferred implementations, persons skilled in the art will recognize that changes may be made in form and detail without departing from the spirit and scope thereof.

What is claimed is:

1. A dentifrice comprising a carrier material, and the dentifrice has a pH between 6 and 8.

2. The dentifrice of claim 1, wherein the carrier material is comprised of one or more of calcium carbonate microsphere, a calcium carbonate nanosphere, a calcium carbonate/hydroxyapatite microplatelets, a calcium hydroxyapatite, or a mesoporous hydroxyapatite particle, and wherein the carrier material is used to carry fluoride ions, eugenol, menthol, or another pharmacon.

3. The dentifrice of claim 2, wherein the carrier material further comprises a phospholipid or a phosphoprotein.

4. The dentifrice of claim 2, wherein the carrier material is casein coated.

5. The dentifrice of claim 2, wherein the pharmacon is doxycycline, chlorhexidine, or prednisolone.

6. The dentifrice of claim 1, wherein the dentifrice is an active ingredient in a toothpaste, a dental floss, a toothpick, a mouthwash, or a coating on a dental device.

7. The dentifrice of claim 1, wherein the dentifrice is an application that is applied to teeth, gums, or an interior surface of an oral cavity.

8. A method of administering a cargo to teeth or gums, the method comprising applying a composition with an active ingredient comprising a carrier material and the cargo, wherein the composition has a pH between 6 and 8.

9. The method of claim 8, wherein the composition is applied to occlude dental tubules, attach to dentin, or attach to gum tissue, or attach to enamel.

10. The method of claim 8, wherein the carrier is one or more of calcium carbonate microsphere, a calcium carbonate nanosphere, a calcium carbonate/hydroxyapatite microplatelets, a calcium hydroxyapatite, or a mesoporous hydroxyapatite particle.

11. The method of claim 10, wherein the carrier further comprises a phospholipid or a phosphoprotein.

12. The method of claim 10, wherein the carrier is casein coated.

13. The method of claim 8, wherein the cargo is fluoride ions, eugenol, menthol or another pharmacon.

14. The method of claim 8, wherein at least a portion of the cargo remains on the teeth or gums for over 10 minutes.

15. The method of claim 8, wherein at least a portion of the cargo remains on the teeth or gums for over 30 minutes.

16. The method of claim 8, wherein at least a portion of the cargo remains on the teeth or gums for at least one hour.

16. A method of claim 11 wherein the casein is digested by a matrix metalloproteinase (MMP) enzyme.

17. A method of claim 16 wherein the MMP enzyme is a matrix metalloproteinase-9 (MMP-9) enzyme.

18. A dentifrice composition comprising a casein-coated calcium carbonate microsphere and a cargo, wherein the dentifrice has a pH of about 6-8 and the cargo is fluoride ions, eugenol, or a pharmacon.
